(19) United States
(12) Patent Application Publication
(10) Pub. No.: US 2008/0187489 A1
(43) Pub. Date: Aug. 7, 2008

(54) Generator and Method for Production of Technetium-99m

(75) Inventors: Richard Tomlison, Ontario (CA); Bruce Collier, Ontario (CA); Alan Guest, Port Severn (CA)

Correspondence Address: GOWLING, LAFLEUR HENDERSON LLP, ONE MAIN STREET WEST, HAMILTON, ON, L8P 4Z5, omitted

(73) Assignee: MCMASTER UNIVERSITY, Hamilton, ON (CA)

(21) Appl. No.: 11/665,186

(22) PCT Filed: Oct. 6, 2005
(86) PCT No.: PCT/CA05/01532

§ 371 Date: Apr. 12, 2007

Related U.S. Application Data
(60) Provisional application No. 60/617,031, filed on Oct. 12, 2004.

(51) Int. Cl. A61K 51/00; C01G 57/00; B01J 19/00

(52) U.S. Cl. 424/1.61; 75/393; 422/159

(57) ABSTRACT

A generator that allows for a non-fission based method of producing and recovering ^{99m}Tc from neutron-irradiated molybdenum. This generator system is based on the isolation of ^{99m}Tc, as the decay product from a source of ⁹⁹Mo labelled molybdenum carbonyl Mo(CO)₆ through a distillation process. The ^{99m}Tc obtained from this distillation is produced with high efficiency and purity in a solvent-free form, which can then be dissolved in water or other solvents to produce a solution at the required specific activity and concentration, as reasonably determined by the operator.

### FIELD OF THE INVENTION

This invention relates to the production of technetium-99m, a radioisotope for diagnostic medical purposes. In particular, the invention provides a method and apparatus for the recovery of technetium-99m from molybdenum 99.

### BACKGROUND OF THE INVENTION

The most commonly used radioisotope for diagnostic medical purposes is ^{99m}Tc. The energy of its radiation is ideal for diagnostic imaging, and its half-life of 6 hours is short enough so that relatively large amounts can be used with low probability of radiation injury. ^{99m}Tc is the radioactive daughter of molybdenum-99 (⁹⁹Mo), which has a half-life of 66 hours. When ⁹⁹Mo is produced, the ^{99m}Tc daughter begins to accumulate, but because of its shorter half-life, it reaches an equilibrium level where there will be approximately one disintegration of a ^{99m}Tc atom for every disintegration of a ⁹⁹Mo atom. In 24 hours (4 half-lives of the ^{99m}Tc), this equilibrium is very nearly reached. If one has a source of ⁹⁹Mo, it is therefore possible to remove an almost equivalent amount of ^{99m}Tc every 24 hours.

Currently, the recovery of ^{99m}Tc from ⁹⁹Mo is by means of an apparatus called a generator, as described in U.S. Pat. Nos. 4,837,110; 4,280,053; 4,020,351. The most prevalent type of generator consists of molybdenum-99 molybdate adsorbed onto an aluminum oxide column; the ⁹⁹Mo undergoes nuclear decay to ^{99m}Tc, which is in the chemical form as the pertechnetate. The ^{99m}Tc is obtained from the column by eluting with a sodium chloride solution. As a consequence of the decay of ⁹⁹Mo, the generator produces less ^{99m}Tc over time, such that by the third day (72 hours), the generator produces less than half the amount it produced on the day it was first received and put in use. Typically, a generator is used for about one week before it is replaced by a new generator. After a few months, the old generators are discarded when the ⁹⁹Mo has decayed to essentially background levels.

The major source of high specific activity ⁹⁹Mo used in these generators is isolated from the fission product mixture obtained from nuclear fission of uranium-235 and has a very high specific activity. When a fissionable atom, such as uranium 235 (²³⁵U) undergoes fission in a nuclear reactor, it splits into two fragments known as fission products. In approximately 6% of such fissions, one atom of ⁹⁹Mo is formed, which is equivalent to 3% of all fission products. Under these conditions, the most economical and efficient preparation of ⁹⁹Mo requires highly enriched uranium (HEU). The use of HEU in this process presents many non-proliferation issues for companies wanting to use this technology. Furthermore, after separation of the ⁹⁹Mo for use in technetium generators, the remaining HEU is contaminated with immense amounts of other fission products. The highly active waste generated by this technology presents serious disposal and storage issues for companies wishing to use this technology.

A feature of the ⁹⁹Mo produced in this fission-based process is that it is relatively free of non-radioactive isotopes of molybdenum and can be conveniently adsorbed on a column that is no bigger than a small pencil. As a result, Curie amounts of ⁹⁹Mo can be loaded on a generator and shielded in a lead container, which is easily transported to a radiopharmacy for dispensing.

Alternatively, ⁹⁹Mo can be produced in commercial quantities by neutron irradiation of ⁹⁸Mo in a nuclear reactor [ref: “Obtaining Mo-99 in the IRT-T research reactor using resonance neutrons”. Ryabchikov, Skuridin, Nesterov, Chibisov, Golovkov; Nuclear Instruments and Methods in Physics Research, B 213, 364 (2004)]. The production rate in a typical high neutron flux reactor yields desired product in the range of 1 Ci/g to 10 Ci/g specific activity using natural molybdenum metal. This yield is highly unfavourable compared to over 10⁴ Ci/g for fission-based production. Even if the separated ⁹⁸Mo isotope, which is approximately 24% in natural abundance, is used, the proportion of ⁹⁹Mo in this target is very low compared to that obtained as a fission product. As a result of the low specific activity, the size of an aluminum oxide column to accommodate this ⁹⁹Mo in a generator would be larger and require greater amounts of shielding. In addition to the shielding and associated handling problems, the volume of sodium chloride solution, time of recovery and efficiency of recovery detracts from the use of this type of non-fission produced technetium generator from an economical and commercial perspective.

Two other types of generator have been proposed for recovering the ^{99m}Tc from irradiated ⁹⁸Mo. When the molybdenum is irradiated in the oxide form (MoO₃), it is possible to distill the ^{99m}Tc from this at temperatures of 800-900° C. However, the efficiency of the ^{99m}Tc recovery is dependent on the size of the target and is also lower for each successive recovery. For commercially acceptable amounts, the recovery time is excessively long in relation to the half-life of the ^{99m}Tc with yields of less than 50%.

A second type of generator that has been extensively studied involves solvent extraction of the ^{99m}Tc. If irradiated MoO₃ is dissolved in KOH to form a solution of K₂MoO₄, the ^{99m}Tc may be extracted from this with methyl ethyl ketone (MEK) provided the ^{99m}Tc is in the pertechnetate state. Various ways of recovering the ^{99m}Tc have been investigated, including evaporation of the MEK and adsorption on alumina. Various subsequent procedures have been utilized to improve the purity of the product ^{99m}Tc, however, MEK produced ^{99m}Tc often gives poor yields when used for the labelling of radiopharmaceuticals.

### SUMMARY OF THE INVENTION

This invention relates to a generator that allows for a non-fission based method of producing and recovering ^{99m}Tc from neutron-irradiated molybdenum. This generator system is based on the isolation of ^{99m}Tc, as the decay product from a source of ⁹⁹Mo labelled molybdenum carbonyl Mo(CO)₆ through a distillation process. The ^{99m}Tc obtained from this distillation is produced with high efficiency and purity in a solvent-free form, which can then be dissolved in water or other solvents to produce a solution at the required specific activity and concentration, as reasonably determined by the operator.

### BRIEF DESCRIPTION OF DRAWING

In order that the invention can be more clearly understood, a preferred embodiment is described below with reference to the accompanying drawing which is a schematic layout of apparatus used to generate technetium-99m from labelled molybdenum carbonyl.

### DESCRIPTION OF THE INVENTION

The generator system of this invention involves a distillation procedure to enable the separation of ^{99m}Tc from ⁹⁹Mo in a closed system with the opportunity to perform multiple recoveries. The recovery time required to isolate the ^{99m}Tc depends on the level of specific activity, but is short compared to the half-life of ^{99m}Tc.

The operation of the generator depends on the distillation of molybdenum carbonyl Mo(CO)₆ labelled with a high specific activity of ⁹⁹Mo hereinafter referred to as labelled molydenum carbonyl. When ⁹⁹Mo in this carbonyl compound decays to ^{99m}Tc, the ^{99m}Tc is not volatile and quantitatively remains in the distillation vessel. It may be recovered from this vessel with any aqueous or non-aqueous solvents at the desired concentration, as determined by the operator. It will be understood that aqueous solutions are desirable for intravenous injection into the human or animal body. The distilled Mo(CO)₆ is recovered in a second vessel where a further ^{99m}Tc recovery can be obtained by a subsequent distillation back to the first vessel, after suitable delay in order to allow for the accumulation of the desired amount of ^{99m}Tc as determined by the needs of the operator.

The production of the labelled Mo(CO)₆ is outside the scope of this invention. In one method, direct irradiation of the Mo(CO)₆ is envisioned, although this is not expected to be the most productive method. When Mo(CO)₆ is irradiated with neutrons in a nuclear reactor, approximately 70% of the ⁹⁹Mo produced is retained as ⁹⁹Mo(CO)₆, a phenomenon known as retention. This irradiated molybdenum carbonyl (⁹⁹Mo(CO)₆) along with the unreacted starting material, can be recovered by distillation. By distillation, it is meant “a process that consists of driving gas or vapour from liquids or solids by heating and condensing to liquid products”. The remaining 30% of the ⁹⁹Mo escapes from the Mo(CO)₆ by the Szilard-Chalmers process and is non-volatile, but is also associated with other non-volatile products related to the decomposition of Mo(CO)₆. To minimize the decomposition products, the target Mo(CO)₆ must be cooled. Alternatively, the irradiation time can be shortened compared to that required to reach the saturation levels of ⁹⁹Mo. Therefore, the specific activity of the ⁹⁹Mo(CO)₆ is reduced by both losses due to decomposition and/or a shortened irradiation time.

Alternatively and more preferably, very high specific activity of ⁹⁹Mo in Mo(CO)₆ can be obtained by direct irradiation of molybdenum metal powder in a nuclear reactor. Subsequent conversion of this irradiated molybdenum to Mo(CO)₆ can be carried out by standard chemical procedures, such as heating the metal to about 225° C. at 200 atmospheric pressure in the presence of carbon monoxide, or other methods as known to those skilled in the art.

A significant feature of the Mo(CO)₆ system is that once the ⁹⁹Mo has decayed to the extent that it is no longer useful in the generator, the residual carbonyl compound can be heated to a temperature above 150° C. to decompose the compound back to molybdenum powder and can be re-irradiated in the nuclear reactor. In this way, separated ⁹⁸Mo used as the target material can be recycled.

### DESCRIPTION AND OPERATION OF THE GENERATOR

Other features and advantages of the present invention will become apparent from the following description. It should be understood, however, that the detailed description and the examples while indicating preferred embodiments of the invention are given by way of illustration only, since various changes and modifications within the spirit and scope of the claims will become apparent to those skilled in the art from this detailed description.

The size of the generator depends on the mass of labelled Mo(CO)₆ to be processed. For example, a 5 Curie generator, which might require only 5 grams of the carbonyl can be relatively small. There is, however, no restriction on the magnitude, and a kiloCurie generator is possible. It should be noted that the size of this generator, with the requisite shielding, could be significantly larger than that used for fission produced ⁹⁹Mo. However, this generator would be reusable and could be permanently located at a central site for distribution of the recovered ^{99m}Tc to surrounding hospitals. In such a scenario, the irradiated Mo metal or the labelled Mo(CO)₆ would then be delivered to the central site. Shielding required for transportation of either of these irradiated products would be similar in size to that used for currently available commercial generators based on fission products.

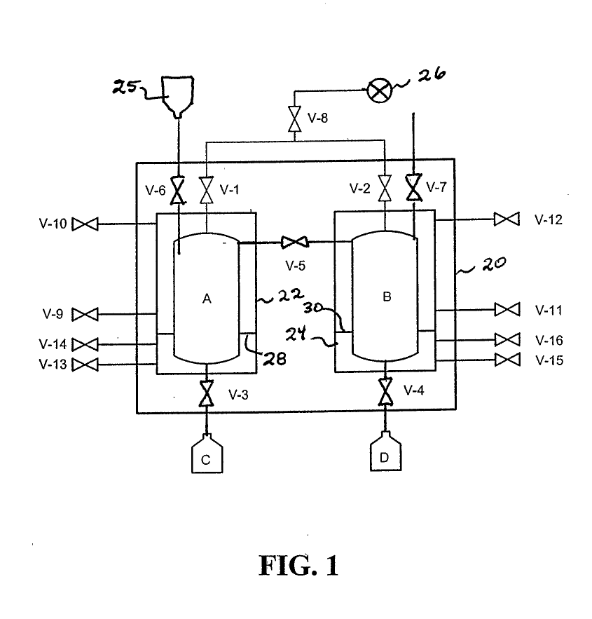

A schematic diagram of one type of generator is shown in FIG. 1, with the following features:
Valves 1 to 7 indicated by reference characters V-1 to V-7 are all remotely actuated and are inside radiation protective shielding 20, whose thickness is determined by the activity of materials used in the generator.
Vessels A and B are identical and may be heated or cooled by surrounding respective envelopes 22, 24, in which appropriate fluids are allowed to flow, such fluids entering and exiting through any combination of valves indicated by reference characters V-9 to V-16, as required. For example, to adjust vessel A to a single temperature, a fluid at that temperature is allowed to enter into the surrounding envelope through valves V-9 and V-13, and exit through valves V-10 and V-14. It is also possible to have two distinct temperatures maintained in the vessel. For example, a hot fluid enters through valve V-13 and exits through valve V-14, while cold fluid is passed through valve V-9 to valve V-10. To more effectively control the temperature in the envelopes 22, 24, an insulated, horizontally oriented partition 28, 30 is disposed in each of the envelopes 22, 24 respectively, thereby separating a heating zone from a cooling zone.
Vials C and D are sterile vials fitted with septa, which will be used to introduce solvent to recover the ^{99m}Tc from vessels A or B.

The labelled Mo(CO)₆ is introduced into the generator from a supply vessel 25 through valve V-6. Vessel A is cooled to at least 0° C. with valves V-3 and V-5 closed and valve V-1 and V-8 open to a vacuum pump 26. The temperature in Vessel A may be reduced further as low as 10° C. to reduce the vapor pressure of carbonyl in the vessel. When all Mo(CO)₆ has been collected in vessel A, valves V-6 and V-1 are closed. After appropriate time to allow build up of ^{99m}Tc, vessel A is heated to a temperature such as 100° C. or higher sufficient to allow rapid distillation of the carbonyl. Vessel B, with valves V-4 and V-7 closed, is evacuated through valve V-2 to dry the vessel, and is subsequently cooled to at least 0° C. Valve V-5 is then opened to allow the distillation of the Mo(CO)₆ into vessel B from vessel A. After this distillation, vessel A will contain only the ^{99m}Tc to be recovered. Valve V-5 and V-7 are then closed. The ^{99m}Tc is recovered from vessel A by filling vial C with an appropriate volume of solvent, such as outgassed water containing a small amount of H₂O₂. The volume selected will be such that when it has extracted the expected amount of ^{99m}Tc, its specific activity will be that required by the user. Vial C is then connected to valve V-3 by a hypodermic needle extending to the bottom of the vial. Valve V-3 is opened to allow the solvent in the vial to be taken up into vessel A, after which valve V-3 is closed. The upper part of vessel A is cooled while the lower part of vessel A is heated to allow the solvent (outgassed water) to reflux in vessel A and collect the ^{99m}Tc at the bottom of the vessel. Such a method of reflux recovery adapted to remove iodine 125 from the interior of a decay chamber in which iodine 125 is formed by decay of Xenon 125 as described in applicant's U.S. Pat. No. 6,056,929, the disclosure of which is herein incorporated by reference. Air is then allowed to enter vessel A through valve V-1 and V-8, and valve V-3 is also opened to allow the solvent containing the ^{99m}Tc to flow in to vial C. After the solution of ^{99m}Tc is collected, valve V-3 is closed and the air and remaining moisture can be pumped out of vessel A through valves V-1 and V-8. After an appropriate time to allow the accumulation of ^{99m}Tc in vessel B, the Mo(CO)₆ is distilled back into vessel A and the recovery of a second yield of ^{99m}Tc can be obtained from vessel B into vial D in a similar manner, as described above.

1. A method of generating a technetium-99m isotope from a source of labelled molybdenum carbonyl Mo(CO)₆ having a predetermined amount of molybdenum-99 isotope in which:
the labelled molybdenum carbonyl is introduced into a first vessel for containing labelled molybdenum carbonyl and allowed to decay for a pre-determined period of time;
the first vessel is heated after said pre-determined period of time to distil labelled molybdenum carbonyl into a second vessel for containing labelled molybdenum carbonyl; and
the technetium-99m isotope remaining in the first vessel after distillation is recovered from the first vessel.

2. A method according to claim 1 in which labelled molybdenum carbonyl contained in the second vessel is allowed to decay for a pre-determined period of time, the second vessel is heated after said pre-determined period of time to distil labelled molybdenum carbonyl into the first vessel, and the technetium-99m isotope remaining in the second vessel after distillation is recovered from the second vessel.

3. A method according to claim 1 in which the labelled molybdenum carbonyl is introduced into the first vessel for containing labelled molybdenum carbonyl through distillation from a supply vessel.

4. A method according to claim 1 in which the labelled molybdenum carbonyl is introduced into the first vessel for containing labelled molybdenum carbonyl through distillation from the second vessel.

5. A method according to claim 1 in which the first vessel is cooled prior to introducing the labelled molybdenum carbonyl into the first vessel.

6. A method according to claim 5 in which the first vessel is cooled to at least 0° C.

7. A method according to claim 6 in which the first vessel is cooled to approximately 10° C. below zero.

8. A method according to claim 1 in which the first vessel is heated to a temperature of at least 100° C. for distillation of the labelled molybdenum carbonyl into the second vessel.

9. A method according to claim 1 for generating a technetium-99m isotope solution in which the technetium-99 isotope remaining in the first vessel is recovered by introducing a solvent into the first vessel to dissolve the technetium-99m isotope and removing the resulting technetium-99m isotope solution from the first vessel.

10. A method according to claim 8 in which the solvent is an aqueous medium adapted for intravenous injection into the human body.

11. A method according to claim 9 in which the solvent is an aqueous medium containing a precursor to form a radiopharmaceutical.

12. A method according to claim 9 in which the first vessel is heated in a lower portion thereof to allow the solvent to reflux in the first vessel and collect the technetium-99m in said lower portion of the first vessel.

13. A method according to claim 9 in which the volume of solvent introduced into the first vessel is selected so that the specific activity of the resulting technetium-99m isotope solution will be suitable for an end user.

14. Generator for producing a technetium-99m isotope from a source of labelled molybdenum carbonyl Mo(CO)₆ having
a first containment vessel in fluid communication with a second containment vessel having first valve means for controlling flow therebetween;
second valve means for coupling the first and second containment vessels to a vacuum pump;
inlet means for introducing labelled molybdenum carbonyl into said first and second containment vessels;
outlet means for recovering technetium-99m from said first and second containment vessels; and
heating and cooling means for controlling the temperature of said first and second containment vessels.

15. Generator according to claim 14 in which said first and second containment vessels are shielded to minimise radiation exposure to users.

16. Generator according to claim 14 in which each said first and second containment vessels has heating and cooling means adapted to perform any one of the following: heat the entire containment vessel, cool the entire containment vessel, and simultaneously heat and cool the containment vessel in discrete portions thereof.

17. Generator according to claim 14 in which each said first and second containment vessels is disposed in a respective envelope having a horizontally oriented partition to separate a heating zone from a cooling zone.

18. Generator according to claim 17 in which the partition is insulated.
